(19) United States
(12) Patent Application Publication
(10) Pub. No.: US 2004/0076623 A1
(43) Pub. Date: Apr. 22, 2004

(54) Activated modular grafted polymeric surfaces

(76) Inventors: Nicholas Jon Ede, East Malvern (AU); Francesca Ercole, Clayton (AU); Gordon Tribbick, Clayton (AU); Saman Sandayake, Wheelers Hill (AU); Senake Perera, Wantirna South (AU)

Correspondence Address: Michael B Stewart, Rader Fishman & Grauer, Suite 140, 39533 Woodward Avenue, Bloomfield Hills, MI, 48304, US

(21) Appl. No.: 10/332,892

(22) PCT Filed: Jul. 13, 2001
(86) PCT No.: PCT/AU01/00850

(51) Int. Cl. A61K 38/43

(52) U.S. Cl. 424/94.1; 424/600; 424/682

(57) ABSTRACT

The present invention relates generally to new surfaces for solid phase chemistry applications, more specifically plastics surfaces modified by graft polymerisation for use in chemical synthesis and/or immobilisation of chemical entities and/or compounds.

In particular the invention relates to an activated modular grafted polymeric surface, which is suitable for use as a reagent for solid phase organic synthesis, or as a reagent for the affinity capture, presentation or preparation of biomolecules such as proteins, oligonucleotides, nucleic acids, peptides, and lectins. The grafted polymeric surfaces of the invention are particularly useful as scavenger reagents in combinatorial synthetic protocols, and as affinity reagents in protein purification and proteomics.

This application is based on and claims the benefit of the filing date of US provisional application No. 60/218,236 filed on 14 Jul. 2000 and U.S. provisional application No. filed on 6 Apr. 2001 (Attorney Docket No. 660097.408P2).

The present invention relates generally to new surfaces for solid phase chemistry applications, more specifically plastics surfaces modified by graft polymerisation for use in chemical synthesis and/or immobilisation of chemical entities and/or compounds.

In particular the invention relates to an activated modular grafted polymeric surface, which is suitable for use as a reagent for solid phase organic synthesis, or as a reagent for the affinity capture, presentation or preparation of biomolecules such as proteins, oligonucleotides, nucleic acids, peptides, and lectins. The grafted polymeric surfaces of the invention are particularly useful as scavenger reagents in combinatorial synthetic protocols, and as affinity reagents in protein purification and proteomics.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Over the past decade, the simultaneous synthesis of large numbers of compounds, either as discrete members of sets, or as mixtures, has become an important tool in the drug discovery process in the pharmaceutical industry. Whether achieved by manual or automated means, via solution phase or solid-supported (solid phase) synthetic methodologies, this new area of chemical sciences is generally referred to as “combinatorial chemistry”. Sets of compounds prepared by these methods are generically known as “combinatorial libraries”.

The rise in the popularity of combinatorial chemistry has paralleled the development of high throughput screening technologies, which now allow hundreds to thousands of assays to be run concurrently, hence taxing the abilities of traditional medicinal chemistry programs to supply new compounds for the drug discovery process. High throughput screening has become viable due to dramatic developments in robotics and computation, coupled with the equally dramatic developments in molecular biology. Over the past few years, large numbers of receptors have been identified and characterised. Furthermore, many receptors can now be expressed on the surface of cells; these cells can be plated out into convenient array formats, hence facilitating automated screening for the ability of test compounds to bind to the receptor and/or modulate the activity mediated by the receptor.

The original “solid phase” used in these methods was beaded polystyrene cross-linked with divinylbenzene. Over the years, polymethylacrylamide (e.g., PEPSYNKT™), polystyrene-polyethylene glycol copolymers(e.g., RAPP TENTAGEL™), macroporous polystyrene (e.g., Polymer Laboratories Stratospheres) and many other “solid phases” have been developed. Many of these “resins” were designed to service the peptide and oligonucleotide synthesis market. However, there are limitations in using such resins, including the need for a containment strategy for synthesis of compounds in quantity, the different reaction profiles between different bead sizes due to increased diffusion pathlength and increased cross-linking with larger resin beads, differential swelling in different solvents, with some resins having 8-fold differences in volume and limited temperature stability, with a maximum of 150° C. for the best performing resins.

Another type of solid phase which has become available more recently is a pellicular type of solid support where a more mobile polymer is grafted to rigid plastics (hereinafter referred to as “grafted supports”). Compared to the resins which dominate this field, grafted supports allow great flexibility of design as plastics are available as sheets, films, threads or can be moulded into any shape as required. Many different polymers or co-polymers can be grafted on to any particular shape to give a wide choice of options in the physicochemical characteristics of the actual solid support. Unlike resins, it is the surface area of the grafted support and not the volume that determines loading capacity. It is possible to achieve consistency of reaction kinetics between grafted supports of different sizes and shapes. This is difficult with resins, because diffusion pathlength must change with size and, as a consequence, reaction rates must change. Other advantages include an integrated design of the solid phase used in synthesis with a method of containment into “resin packets” and minimal swelling in different solvents as the majority of the substrate polymer does not swell.

The limitations with the current grafted supports include the temperature limit of about 120° C., the loading per unit volume is lower than the best high loading resins and the perceived reaction rate is slower than conventional resins.

Traditionally, pharmaceutical companies have screened their large compound inventories, which have been built up over many years, to discover new lead compounds. These inventories have been built up from a number of sources, including natural products and synthetic intermediates. However, it has been the labour of large numbers of medicinal chemists, working over many decades, which has been largely responsible for the compound collections. Medicinal chemists have generally prepared small sets of compounds within a target family and obtained preliminary screening data using a limited range of screening assays before proceeding to make further compounds in that family.

With its ability to provide more compounds per unit time and per unit chemist, and with the increasing difficulty of finding new lead compounds, combinatorial chemistry is becoming increasingly widely utilised, using either solution phase or solid phase techniques. Hence the number of compounds produced by a typical chemist in a year may be increased by orders of magnitude.

Combinatorial chemistry represents an important paradigm shift in the practice of synthetic chemistry. Over the past few years, enormous advances have been achieved in the ability of chemists to enhance the synthesis and design of molecules which fit a specific set of parameters, rather than just indiscriminately and wastefully synthesising a vast number of compounds. Currently, there are two schools of thought as to the scale of synthesis required for library synthesis. While there is no doubt that the trend in compound library generation is towards well-designed, individual, pure and fully characterised compounds, there is still a split in the,.scale of synthesis between large scale (20 mg/compound) and small scale (0.5-1 mg/compound) syntheses.

Despite the success and advantages of solid phase organic chemistry, there are still limitations to this technique. Firstly, the reactions can be slow relative to their solution phase counterparts, although the development of high-performance polyethylene glycol-based resins and grafted polymeric supports has alleviated this problem to a certain extent.

Secondly, it can also be difficult to monitor reaction progress. Considerable improvements have been made in this area in recent years, with the use of techniques such as Fourier transform infra-red spectrometry, matrix-assisted laser desorption inteferometry-mass spectrometry, Gelphase and Magic Angle Spinning (MAS) nuclear magnetic resonance spectrometry etc. However, these techniques still do not provide the same quality of analysis as rapidly and conveniently as conventional solution-phase techniques such as thin layer chromatography, gas chromatography-mass spectrometry, liquid chromatography-mass spectrometry, SFC-mass spectrometry, nuclear magnetic resonance spectrometry etc. This problem has been partly addressed recently with the use of grafted polymer surfaces in MAS-nuclear magnetic resonance monitoring of solid phase reactions (Sefler and Gerritz, J. Com. Chem. 2000, 2, 127-133).

Thirdly, one of the most time-consuming aspects of solid-phase organic chemistry is usually the process of attempting to optimise solution-phase chemistry on a polymer-supported substrate, particularly where a long synthetic sequence is required.

Over the last few years increased emphasis has been placed on the development of parallel solution phase methods of combinatorial library synthesis. Solution-phase synthesis has obvious advantages: experience gained over 100 years of chemical reaction experience is available, reactions can be monitored using standard techniques, and no additional steps are required to attach and detach compounds from the solid support.

In the past, solution-phase parallel synthesis was usually only applied to short, high-yielding reaction sequences, because of the difficulties in purifying large numbers of reaction products simultaneously. These problems have been recently addressed, with the development of specifically-functionalised resins designed to react with and remove unreacted reagents from a reaction mixture. These so-called polymer-supported reagents and scavenger resins have revolutionised solution-phase parallel synthesis.

Supported reagents are reactive species which are associated with a support material. They transform a substrate(s) to a new chemical product(s), and the excess or spent reagent may be removed by filtration.

Supported catalysts are reactive species which are associated with a support material. They are used in sub-stoichiometric quantities to transform a substrate(s) to a new chemical product(s), and may be removed by filtration and recycled

Supported scavengers are reactive species which are associated with a support material. They selectively quench or sequester by-products of the-reaction, or remove excess or unreacted starting materials, and may be removed by filtration.

In this way the advantages of solid-phase synthesis, ie the ability to use excess reagents to drive reactions to completion, and the ease of product isolation, have become applicable to solution-phase synthesis. A crucial advantage for industrial processes is that toxic, noxious or hazardous reagents and their by-products can be immobilised, and are therefore not released into solution, thereby improving their general acceptability, utility and safety profile. In addition, for reactions which are low-yielding and result in complex mixtures of products, scavengers can be used to isolate pure products in: a simple fashion, without the need or stedious conventional work-up and purification procedures. Simple work-up procedures such as filtration and solvent removal make solid-supported reagents and scavengers particularly attractive for combinatorial library generation.

As the number of compounds to be synthesised increases, so too does the complexity of handling the resins in parallel. Thus a fundamental limitation to this method is that the solid phase needs to be removed in a high-throughput parallel fashion, thus negating some of the advantages of using resins. Expensive equipment such as robotic automation is generally required to remove the resin. In addition, because resins swell in solvent, large excesses of solvents are required to eliminate the possibility of the reaction solutions becoming essentially dry. Organic solvents may be toxic, and their disposal presents a significant environmental problem.

Affinity separation of molecules, whether large or small, requires a fundamental understanding of how the molecule(s) of interest interact with the solid-phase matrix. These fundamental interactions are all chemically based, and involve the interaction of chemical functional groups. An understanding of how different functional groups interact between the solid and solution phase enables the prediction of how specific proteins will react with specific solid-phase surfaces.

We have now found that certain specific plastics and modified forms thereof can be used as a solid support for multiple parallel organic compound synthesis and several other related applications.

We have also found that significantly improved efficiency of synthesis can be achieved by using reagents and scavengers bound to a solid-phase support which is a modular grafted polymeric surface. The use of a modular grafted surface overcomes both the disadvantages listed above. The modular grafted surfaces of the invention are applicable both to general solid phase organic synthesis and to the affinity capture, presentation or preparation of biomolecules such as proteins, oligonucleotides, nucleic acids, peptides, and lectins.

### SUMMARY OF THE INVENTION

In brief, this invention is generally directed to an activated modular grafted polymeric surface, which is suitable for use as a reagent for solid phase organic synthesis, or as a reagent for the affinity capture, presentation or preparation of biomolecules such as proteins, oligonucleotides, nucleic acids, peptides, and lectins. The grafted polymeric surfaces of the invention are particularly useful as scavenger reagents in combinatorial synthetic protocols, and as affinity reagents in protein purification and proteomics.

In a first aspect, the invention provides an activated modular grafted polymeric surface.

The term “grafted polymeric surface” refers to a polymer which has been modified by graft polvmerisation. Derivatives, blends and copolymers thereof modified by graft polymerisation are also within the scope of the invention. A very wide variety of base polymers may be used, for example an optionally-substituted polyolefin, silicone polymer, natural or synthetic rubber, polyurethane, polyamide, polyester, formaldehyde resin, polycarbonate, polyoxymethylene, polyether, or epoxy resin, or a co-polymer comprising any of these. Optionally-substituted polyolefins may be selected from polyalkenes, such as polyethylene, polypropylene, and polyisobutylene; acrylic polymers, such as polyacrylate, polymethacrylate, and polyethylacrylate; vinyl halide polymers, such as polyvinylchloride; fluoropolymers, such as polytetrafluoroethylene, chlorotrifluoroethylene and fluorinated ethylene-propylene; polyvinylethers, such as polyvinyl methyl ether; polyvinylidine halides, such as polyvinylidine fluoride and polyvinylidine chloride; polyacrylonitrile; polyvinylketones; polyvinyl aromatics; and polyvinyl esters, such as polyvinylacetate. Other suitable base polymers will be known to those skilled in the art, see for example Hans Domininghaus, Plastics for Engineers: Materials, Properties, Applications ( Hanser Publishers, New York, 1992). The skilled person will readily be able to test whether a given polymer is suitable for use in the invention, using routine methods.

The term “solid phase chemistry” is used herein in its broadest sense, and refers to the use of solid supports which are insoluble materials to which chemical entities and/or compounds attach during various chemical applications.

In one preferred embodiment the polymer is a co-polymer of polyethylene and polypropylene. In a second preferred embodiment the polymer is a branched polyolefin, or a derivative, blend or copolymer thereof modified by graft polymerization. This is referred to herein as a “modified branched polyolefin”; this is particularly preferred when the graft is polyacrylic acid. This modified branched polyolefin is used per se and represents a second aspect of this invention.

Preferably the branched polyolefin is a polyalkylalkene, more preferably poly-(4-methylpentene-1), referred to herein as “PMP”. The trade name for PMP manufactured by Mitsui Chemicals Inc(3-2-5 Kasumigaseki Chiyoda-ku, Tokyo 100, Japan) is TPX.

Suitable types of graft polymerisation include gamma-irradiation graft polymerisation, ozone-induced graft polymerisation, plasma-induced graft polymerisation, UV-initiated graft polymerisation and chemical-initiated graft polymerisation, such as peroxide-initiated graft polymerisation.

The polymers which may be grafted on to the branched polyolefin include polyvinyls, polystyrenes, poly-α-methylstyrenes, polyvinylalcohols such as polyvinyl acetate, polyacrylates such as polyacrylic acids, polymethacrylates such as 2-hydroxyethyl methacrylate (HEMA), polyacrylamides such as dimethylacrylamide (DMA), polyethylkene glycols, polylactic acids, and derivatives, blends and copolymers thereof. In one preferred embodiment the graft polymer is a polystyrene or a derivative of α-amethylstyrene. In a second preferred embodiment the graft polymer is a polyvinylalcohol.

The term “activated” refers to a grafted polymeric surface to which is bound a reagent such as triphenylphosphine, a reductant or oxidant, a chelating metal such as nickel or calcium, a scavenger such as a nucleophilic group, e.g. aminomethyl or hydrazino, or an electrophilic group, e.g. isocyanate, tosyl chloride, or benzaldehyde, or a catalyst such as dimethylaminopyridine. A chelating metal is preferred when the graft is polyacrylic acid.

The term “modular” means that the activated grafted polymeric surface is in the form of a plurality of physical units which are suitable for use in a set of simultaneous chemical reactions, and which provide reproducible chemical properties. These may be of a wide variety of desired shapes, such as lanterns, gears, pins, pucks, discs, beads, microtitre plates, sheets, etc. It will be appreciated that the activated grafted polymer may be moulded into any shape, depending on the desired application. This also provides flexibility in the physiochemical properties of the activated grafted polymeric support, and means that a specialised containment apparatus is not required, in contrast to the use of resins.

The activating moiety may be aldehyde, carboxylate, amino, hydroxide, biotin, thiol, tosyl acid, tosyl chloride, hydrazino, isocyanate, or any other chemical moiety which could be used with appropriate chemistry to act as a chemical scavenger, solid-supported reagent, solid-supported catalyst, or affinity capture agent for proteins. Preferably the activating moiety is aldehyde.

Preferably the grafted polymeric surface is able to bind a target agent which is anamine compound capable of Schiff base formation. More preferably the target agent is a biotinylated molecule, such as a peptide, protein, oligonucleotide, lipid or sugar. Alternatively the target agent is a protein, such as streptavidin, or an enzyme, for example horseradish peroxidase.

Optionally there may be one or more spacer sequences, which may be the same or different, between the aldehyde and the derivatised polymer support.

In particularly preferred embodiments, the invention provides

(a) a benzaldehyde polystyrene lantern, which can be used for example to scavenge phenylhydrazine;

(b) a benzaldehyde polystyrene lantern, coupled to streptavidin or horseradish peroxidase; and

(c) a nickel-chelating polyacrylic acid gear, which can be used for affinity separation of hexahistidine-tagged proteins.

It will be appreciated that the protein affinity capture agents of the invention are particularly suitable for use in in proteomics applications.

For the purposes of this specification it will be clearly understood that the word “comprising” means “including but not limited to”, and that the word “comprises” has a corresponding meaning.

### BRIEF DESCRIPTION OF THE FIGURE

FIG. 1 is a diagram showing the dimensions of the PMP Gears.

FIG. 2 is a photographic representation of nickel-chelated polyacrylic acid gears according to the invention (PMP gears), showing from left to right

(1) Gear grafted with acrylic acid (2)

(2) Gear as in (1) stained with Rhodamine B dye,

(3) Gear as in (1) doped with nickel chelating metal and

(4) Gear as in (3) stained with 1% rubeanic acid.

### DETAILED DESCRIPTION OF THE INVENTION

The invention will now be described in detail by way of reference only to the following non-limiting examples and drawings.

In addition to the ability of the grafted polymeric surfaces of the invention to provide flexibility of modular shapes, other advantages of the polymeric surfaces of the invention, particularly the branched polyolefin, include:

high heat-resistance, with a melting point of about 220° C. to about 240° C., which means that it can withstand reaction temperatures up to about 200° C.; this covers the temperature range used in most organic chemistry applications; excellent transparency, with its transmittance of visual light being >90% and of ultraviolet light being about 300 nm to about 400 nm; this makes it useful in applications relying on colour;

excellent chemical resistance;

good heat aging and resistance to steam or boiling water;

high loadings of up to about 300 nmol per sq cm, resulting in superior yield properties compared to resins, where the maximum loading possible is about 124 nmol per sq cm;

suited to both batch and flow type synthesizers; and

when moulded into microtitre plates, has the potential for use in small scale (<500 nmol) synthesis of small molecules (MW<500), peptides and oligonucleotides.

There are two advantages to solid-phase chemistry. Firstly, isolation of support-bound reaction products is accomplished simply by washing away reagents from the support-bound material, and therefore reactions can be driven to completion by the use of excess reagents. Secondly, innovative methods are available for the manipulation of discrete compounds and for tracking the identity of compounds when compounds are attached to a solid support.

Examples of solid phase chemistry applications in which the polymers of the invention are particularly useful include chemical synthesis, scavenging, purification, immobilisation and/or chelation.

The use of grafted surfaces for preparation of solid phase reagents and scavengers presents significant advantages, since

(a) Grafted surfaces can be moulded into many different and desirable shapes, such as lanterns, gears, pins, pucks, beads, discs, microtitre plates, sheets, etc, which provide reproducible chemical properties;

(b) The modular support does not swell, and therefore the volume of reaction solution can be confidently set prior to chemistry being performed;

(c) Modular supports such as lanterns, gears, pins, discs, or pucks can be mounted in an 8×12 or similar format and simply lowered into a reaction solution, allowed to react, and then lifted clear of the solution, in a simple but parallel fashion.

The chemical synthesis applications include synthesis of organic compounds, in particular biological organic compounds such as peptides, proteins and oligonucleotides.

The higher loading capacity of the polymers of the invention, particularly the branched polyolefin and the modified branched polyolefin of the invention, means that they are suitable for use in scavenger applications, i.e. the removal of unwanted by-products from chemical reactions (Thompson, L. A., Recent Opinions in Chemical Biology, 2000, 4, 324-337). Specialist filtration equipment is also not required in order to perform the scavenger reactions using the grafted modular polymeric supports of the present invention.

Another important application is as a solid phase fluorescence quenching assay for use in determination of substrate as well as inhibitor specificity of proteolytic enzymes (St Hilaire, P. M., Willert, M., Juliano, M. A., Juliano, L., Meldal, M. , J. Comb. Chem., 1999, 1, 509-523).

The high loading coupled with the high kinetics obtained from the grafted modular polymeric supports of the present invention makes the polymer an excellent support for affinity chromatography applications. Purification of proteins from proteomics mixtures and purification of DNA from genomic mixtures are two broad areas of application. For example, grafting of polyacrylic acid to branched polyolefin gives a surface which can chelate nickel ions. This surface can then be used to affinity capture proteins which have been genetically engineered to possess a multi-histidine tag such as hexa-histidine. The histidine tag coordinates with the nickel surface.

Branched polyolefin grafted with acrylic acid results in a surface bearing carboxylic acid groups. This surface binds not only metals such as nickel, as described above, but also other metals, for example, copper, zinc, cadmium, silver, palladium, platinum, gold and lead. Thus the modified branched polyolefin has application in removal of metals from the environment, for example in the cleaning of contaminated sites, or in the prevention of pollution, for example in the treatment of industrial waste water before release into effluent streams or in the treatment of sewage.

Although this invention is specifically described with reference to the use of particular modular shapes and combinations of polymer and activating groups, it will be clearly understood that lanterns, gears or other modular shapes activated by other methods, such as carboxylate, amino, hydroxide, biotin, or any other chemical moieties which could be used with appropriate chemistry either as solid phase reagents or scavengers, or as affinity surfaces for capture, presentation or preparation of biomolecules. such as proteins,oligonucleotides, nucleic acids, peptides, and lectins, are suitable for use in the invention.

For many years we have used a multipin array system for solid-phase combinatorial peptide synthesis. This system is marketed by Mimotopes Pty Ltd, Clayton, Australia, and is used for synthesising peptides and for synthesising peptide libraries. The proprietary pin, Crown™ and SynPhase™ Lantern support systems utilise polyethylene or polypropylene copolymers grafted with 2-hydroxyethyl methacrylate polymer (HEMA), methacrylic acid/dimethylacrylamide polymer(MA/DMA) or polystyrene (PS) (see for example Maeji et al., Reactive Polymers 1994, 22, 203-212).

Examples of reagents which may be used in the invention and the purposes for which they are particularly suitable are shown in Table 1. It will be clearly understood that this list is representative only, and is not intended to be exhaustive.

TABLE 1
Grafted Polymer-bound Reagents and Scavengers

| | Compound | |
|---|---|---|
| Structure | Designation | Type |
| 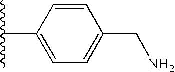 | 1 | Scavenger nucleophilic |
| 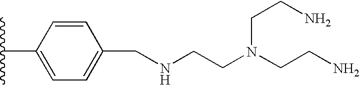 | 2 | Scavenger nucleophilic |
| 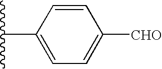 | 3 | Scavenger electrophilic |
| 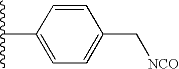 | 4 | Scavenger electrophilic |
| 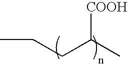 | 5 | Metal chelation surface |
| 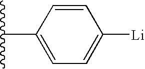 | 6 | Reagent Precursor to numbers 7, 8, 11 and 14 |
| 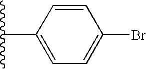 | 7 | Reagent Precursor toll and 14 |
| 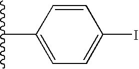 | 8 | Reagent Precursor to 11 and 13 |
| 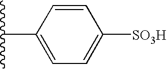 | 9 | Reagent Ion-exchange |
| 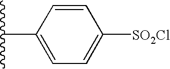 | 10 | Scavenger electrophilic |
| 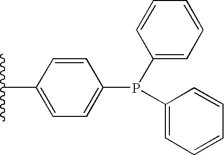 | 11 | Reagent |
| 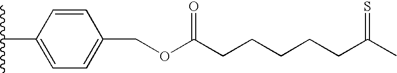 | 12 | Reagent |
| 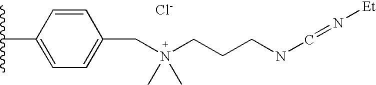 | 13 | Reagent |
| 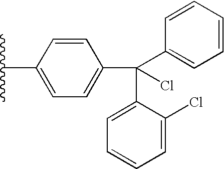 | 14 | Linker |
| 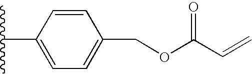 | 15 | Scavenger electrophilic |
| 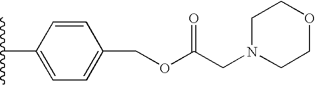 | 16 | Reagent |
| 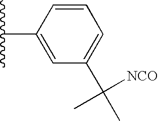 | 17 | Scavenger electrophilic |

### EXAMPLE 1

### Grafting of 100 PMP “Gears”*

Styrene (48 mL was mixed with methanol (112 mL). The mixture was them pored into a bottle containing 1000 Gears. The mixture was purged with nitrogen for 20 minutes. The bottle was capped tightly and them then placed in the irradiator machine. The contents were irradiated at a dose rate of 1.6 KGy per hour for 7 hours with occasional shaking of the bottle. The bottle was removed, the styrene drained and the contest washed with dichloromethane (5×10 mimutes). The gears were removed and air dried before being derivatised.

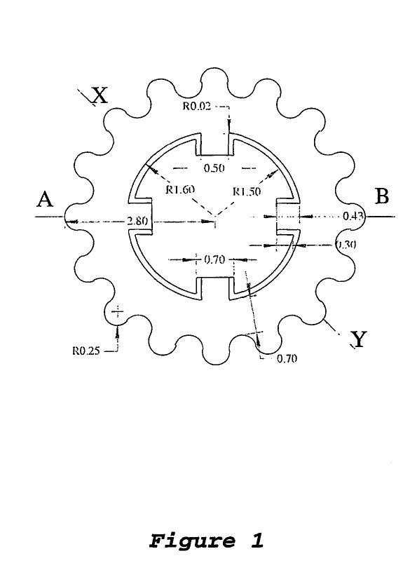

*“Gears” refer to a specific moulded shape of PMP. See FIG. 1 for dimensions.

### EXAMPLE 2

### Solid Phase Synthesis of 4-Chloro-3-[3-(4-trifloromehyl-phenyl)-ureido]-benzamide

The grafted polystyrene gears of Example 1 were aminomethylated and derivatised wiht a Rink linker, using standar methods as described by Adams et al Adams, J. H., Cook R. M., Hudsen, D., Jammalamadaka, V., Lyttle, M. H., and Songster, M. F., J. Org. Chem., 1998, 63, 3706-3716), and shown in Scheme 1 below.

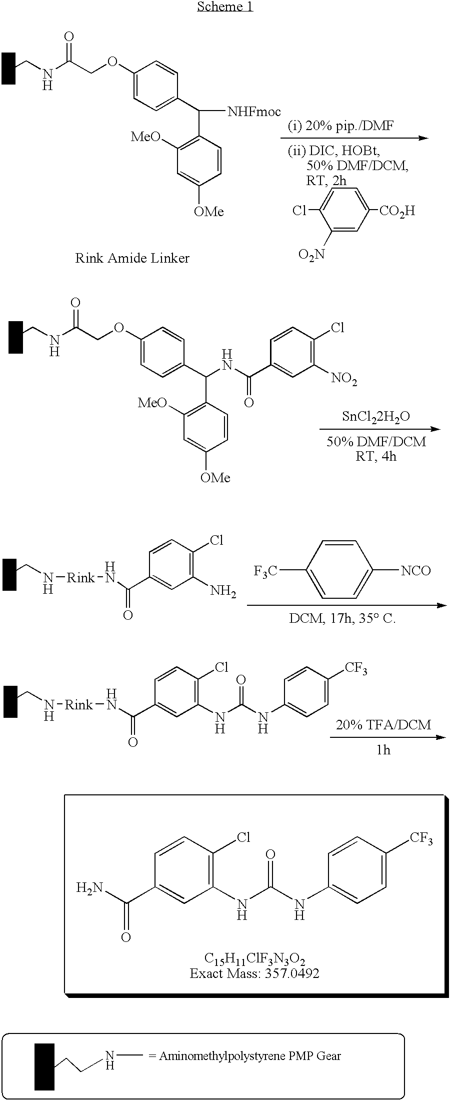

Gears (loading=9 μmol) were covered with 20% v/v piperidine in DMF and stood at room temperature for 30 mins. The solution was decanted and the gears washed in turn with DMF (2×3 mins) and DCM (2×3 mins). Each Fmoc-deprotected gear was treated with 0.5 mL of a solution of DIC (0.2 M, 100 μmol, 2.9 mole equivalents), HOBt (0.2 M, 100 μmol, 2.9 mole equivalents) and 4-chloro-3-nitrobenzoic acid (0.2 M, 100 μmol, 2.9 mole equivalents) in 50% v/v DCM/DMF at room temperature for 2 h. The solution was decanted and the gears washed in turn with DMF (2×3 mins) and DCM 2×3 mins). Each-gear was treated with 0.5 mL of a 2 M solution of tin (II) chloride dihydrate (1000 μmol, 29 mole equivalents) at room temperature for 4 h. The solution was decanted and the gears washed in turn with DMF (2×3 mins) and DCM (2×3 mins). Gears were pre-dried in a vacuum oven at 40° C./approximately 1 mmHg for 18 h. Each gear was treated with 0.5 mL of a 0.5 M solution of 4-trifluoromethylphenylisocyanate (250 μmol, 7.4 mole equivalents) in anhydrous DCM at 35° C. for 18 h. The reaction was allowed to cool to room temperature and the solution decanted. The gears were washed in turn with DCM (1×3 min), DMF (2×3 min) and DCM (2×3 min) and air dried. Individual gears were placed in Labsystems™ 1.1 mL polypropylene tubes and treated with 20% v/v TFA in DCM (0.6-0.8 mL) for 1 h. The excess TFA and solvent were removed using a centrifugal evaporator. Further drying was achieved by placing in a vacuum oven at 40° C./approximately 1 mmHg for 18 h. A ‘quantitative’yield of 4-Chloro-3-[3-(4-trifluoromethyl-phenyl)-ureido]-benzamide was obtained, based on the weight of crude product and the specified initial loading of the gear. Samples were dissolved in 90% v/v CH₃CN/H₂O for HPLC or Electrospray mass spectrometry (ESMS) analysis and DMSO-d6 -for ¹H NMR spectroscopy. Purity (>95% at 214nm),based on reverse phase HPLC (C18 column). Analytical HPLC was performed on a Waters chromatography system using a Ranin microsorb-mv (#86-200-F3) RP-18 column (100A, 3 μm). The following conditions were used: buffer A=water (0.1% H₃PO₄); buffer B=90% acetonitrile/10% water (0.1% H₃PO₄); linear gradient A to B from 1 to 11 min; flow rate=1.5 mL min⁻¹. Absorbances were recorded at 214 and 254 nm. HPLC purities were determined by peak area at 214 nm. ESMS: found 358.00 [M+H]; expected 358.00. ESMS analyses were performed on a Perkin Elmer Sciex API III mass spectrometer.

### EXAMPLE 3

### Synthesis of Polystyrene-Benzaldehyde Lanterns 3

Fresh dichloromethane (850 mL) was added to polystyrene lanterns (4000) in a 2 L multi-necked round-bottomed flask, followed by dimethyl methyl ether (27.3 mL, 30 mM). Dimethyl methyl ether is highly toxic, and should only be used in a fumehood. Standard precautions must be taken in washing and disposing of equipment and reagents. The contents of the flask were mixed with mechanical shaking under a nitrogen atmosphere. SnCl₄ (15.6 mL, 150 mM) was slowly added using a dry glass syringe. SnCl₄ is highly air sensitive, and should be bubbled with nitrogen before use. The mixture was reacted for 5 hours at room temperature, and drained from the lanterns into a beaker containing methanol (1 L). The lanterns were washed with dichloromethane (2×10 mins), followed by hot 20% H₂O/THF (1 hour), and soaked in 20% H₂O/THF overnight, then soaked in dichloromethane (30 min), and air dried. The product is polystyrene benzaldehyde lanterns 3.

### EXAMPLE 4

### Use of Polystyrene Benzaldehyde Lanterns to Scavenge Phenylhydrazine

Polystyrene benzaldehyde lanterns made as described in Example 3 (10 lanterns, 3 equivalents)) were added to a mixture containing phenylhydrazine (1 equivalent) in 1% acetic acid/ dichloromethane. The mixture was gently agitated for 1 hour at room temperature, after which thinlayer chromatography (solvent system: ethyl acetate, petrol, methanol: 10, 30, 5) indicated complete removal of the phenylhydrazine from the reaction mixture had occurred, yielding 2.

### EXAMPLE 5

### Coupling Proteins to Benzaldehyde Polystyrene Lanterns

The following is a further application of lanterns. A generic procedure for the coupling of proteins to lanterns is presented. As an illustration of the application two proteins were used, streptavidin (SA), and horseradish peroxidase (HRP). The proteins were coupled to the lantern by reaction of the aldehyde with each protein's amine groups to form Schiff's bases. Subsequent reductive amination forms a stable covalent link, with minimal leakage of the protein from the lantern.

In this example various derivatised lantern formats may be used; for example, there may be different spacer sequences between the aldehyde and the derivatised polymer support, and in this example the term lantern will be understood to mean a lantern chemically derivatized to have an aldehyde moiety on the surface. A typical loading for a lantern is ≧15 μmole/lantern.

To demonstrate that protein could be bound to lanterns, the binding of the chosen proteins was tested on untreated lanterns as well as on lanterns treated to chemically block available aldehyde. Because of the nature of protein binding, it is also possible that protein may also bind to nominally blocked surfaces by non-specific absorption. To check for this non-specific binding, blocked lanterns were also prepared and tested.

Blocking Procedure

Several lanterns were incubated for 1 hr at room temperature in a 1 M Tris/HCl buffer pH 7.4 containing 0.2% w/v sodium cyano borohydride (NaBH₃CN).

The treated lanterns were then washed 5 times with water, and once with 0.01 M phosphate buffered saline, pH 7.4 (PBS).

Protein Coupling

Two protein solutions were prepared, streptavidin (0.05 mg/mL containing with 0.2% w/v NaBH₃CN), and horseradish peroxidase (0.05 mg/mL containing 0.2% w/v NaBH₃CN). Lanterns and blocked lanterns were added to these protein solutions, and incubated for 2 hr at room temperature and then overnight at 4° C. All treated lanterns were then washed 5 times with water and once with PBS.

### EXAMPLE 6

### Testing of Protein-Treated Lanterns

The streptavidin-treated lanterns were tested using an Enzyme Linked ImmunoSorbent Assay (ELISA). A monoclonal antibody for which biotinylated positive and negative binding peptides are available was used. Treated lanterns were divided into two groups, one of which was incubated in a PBS solution containing the “positive peptide” and the other in a PBS solution containing the “negative peptide”. The ELISA was according to the biotinylated peptide ELISA protocol described by Tribbick (Immunological Methods Manual 1996, Ch. 10.8, 816-826).

The average absorbance results are shown in Table 2.

TABLE 2
Results of ELISA Assay for Streptavidin

| Lantern-SA-peptide | Absorbance |
|---|---|
| Positive peptide | 1.2 |
| Negative peptide | 0.2 |

The horseradish peroxidase-treated lanterns were washed five times with water, and then individually placed into an aliquot of ELISA substrate. The protocol used was as described above. The average absorbance results are shown in Table 3.

TABLE 3
Results of ELISA Assay for Horseradish Peroxidase

| Lantern | Absorbance |
|---|---|
| Lantern-HRP | 1.35 |
| Blocked Lantern-HRP | 0.78 |

The results obtained for the two proteins illustrate the use of derivatized lanterns for the immobilization of proteins. The results obtained for HRP clearly show that enzyme activity was maintained, and that the protein was not denatured in the binding procedure.

The retention of functional streptavidin activity clearly indicates that the derivatized lanterns can be used to immobilise any biotinylated molecule, such as peptides, proteins, oligonucleotides, lipids and sugars. The use of the lanterns to immobilise streptavidin, avidin or any modified avidin, and in turn to immobilise biotinylated molecules, can be exploited in many applications by those skilled in the art.

### EXAMPLE 7

### Synthesis of Nickel-Chelated Polyacrylic Acid Gears

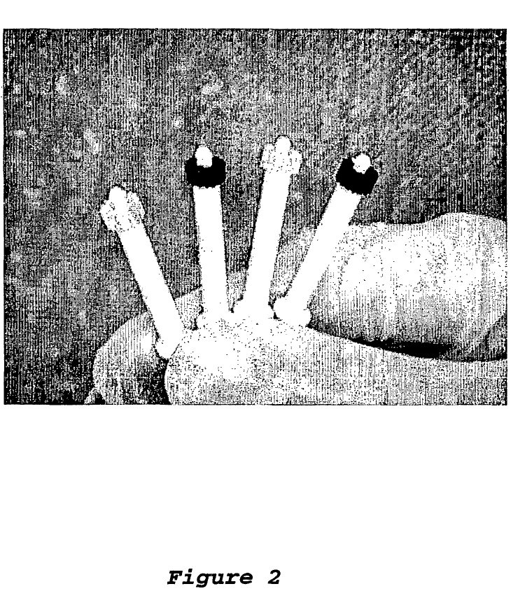

Gears composed of polyacrylic acid-grafted poly-(4-methylpentene-1) were washed with water, 0.1 M NaOH, water and then dosed with nickel solution (150 mM NiSO₄.6H₂O) for 30 minutes. The gears were then washed with water three times. This results in a pale green-coloured gear. To confirm chelation of nickel to the polyacrylic acid graft, nickel-chelated gears were washed with 0.1 M aqueous ammonium hydroxide and then reacted with 1% rubeanic acid in ethanol solution for 60 minutes. After washing with methanol, the gears remain black in colour. This is illustrated in FIG. 2.

### EXAMPLE 8

### Immobilisation of hexa-Histidine Tagged Recombinant Protein on Nickel-Chelating Polyacrylic Acid Gears Containing Different Base Polymers

Three different injected moulded base polymers were assessed for their ability to graft acrylic acid and for their ability to immobilise a hexa-His tagged recombinant protein, SP22 (sperm protein 22) (Welch, J. E., Barbee, R. R., Roberts, N. L., Suarez, J. D., Klinefelter, G. R., J. Androl., 1998, 19, 385-393).

The three base polymers were

(A) poly-(4-methylpentene-1), otherwise known as “PMP”;

(B) a copolymer of polypropylene and polyethylene, otherwise known as “PMA”; and

(C) high-density polyethylene, otherwise known as “HDPE”.

All were grafted with acrylic acid acid and doped with nickel as described in Example 5 to obtain 6% polyacrylic acid gears.

In a 96 well plate, 200 μL of 8 M urea were added to each well, followed by 20 μL of hexa-histidine labelled SP22 dissolved in 8 M urea. The solutions were diluted by 11-fold into two consecutive wells. The three different types of acrylic acid-grafted gears described above were incubated in a 0.1 M solution of NiSO₄.6H₂O for one hour before being washed with water (×2). The nickel-coated acrylic acid gears were placed in the wells containing the diluted solutions of hexa-histidine labelled SP22.

Reaction was allowed to proceed for 1 hour, after which the gears were removed and washed with water (×2). The gears were then placed in wells containing 200 μL of a solution of antibody to hexa-Histidine SP22 (ELISA substrate). The ELISA protocol was according to the protocol described by Tribbick (op. cit). The average absorbance results are shown in Table 4.

TABLE 4
Results of ELISA Assay for hexa-histidine SP22

| | Volume of | | | |
|---|---|---|---|---|
| | Recombinant | | | |
| Type | Protein | | Acrylic Acid | |
| 6% Acrylic | Solution | Ni²⁺⁻coated | Gears | |
| Acid | (μL) | gears | (no nickel) | Difference |
| PMP gears | 18.2 | 2920 | 1504 | 1416 |
| | 1.65 | 1689 | 816 | 873 |
| | 0.15 | 1169 | 743 | 426 |
| PMA gears | 18.2 | 3025 | 3127 | −102 |
| | 1.65 | 2231 | 2107 | 124 |
| | 0.15 | 1647 | 1398 | 249 |
| HDPE gears | 18.2 | 3061 | 2834 | 227 |
| | 1.65 | 1653 | 1542 | 111 |
| | 0.15 | 1222 | 1156 | 66.5 |

The ELISA results clearly demonstrate the superiority of the PMP base polymer for performance of nickel chelating polyacrylic acid gears.

### EXAMPLE 9

### Synthesis of Calcium-Chelating Polyacrylic Acid Gears 5

Gears composed of :polyacrylic acid grafted poly-(4 methylpentene-1) are washed with water, 0.1 M NaOH, water and then dosed with calcium solution (150 mM CaCl₂) for 30 minutes. The gears are then washed with water three times. To confirm chelation of calcium to polyacrylic acid graft, calcium-chelated gears are washed with 0.1 M aqueous ammonium hydroxide and then reacted with 1% glyoxal-bis(2-hydroxyanil) in 0.1 M aqueous ammonium hydroxide solution for 60 minutes. After washing with water, the gears remain red in colour.

### EXAMPLE 10

### Use of Calcium Chelating Polyacrylic Acid Gears for Affinity Purification of Mitochondrial Calcium-Binding Protein

Approximately 100 μL of frozen pelleted mitochondria are suspended in 800 μL of 10 mM Tris-acetate pH 7.0. Two μL of protease inhibitor cocktail (Sigma) are added to the mitochondrial preparation, and the preparation is incubated with calcium-chelating polyacrylic acid gears, prepared as described in Example 9, for 1 hour. The gears are removed and washed with 10 mM Tris-acetate pH 7.0 buffer. The bound calcium-binding proteins are eluted from the gears with 8 M urea buffer.

### EXAMPLE 11

### Synthesis of Concanavalin A-Coupled Lanterns

Ten benzaldehyde polystyrene lanterns are incubated for 1 hr at room temperature in a 1 M Tris/HCl buffer pH 7.4 containing 0.2% w/v sodium cyano borohydride (NaBH₃CN)and Concanavalin A (0.05 mg/mL).

The treated lanterns are then washed 5 times with water, and once with 0.01 M phosphate-buffered saline, pH 7.4 (PBS).

### EXAMPLE 12

### Use of Concanavalin A-Coupled Lanterns for Affinity Purification of Mitochondrial Glycoprotein

Approximately 100 μL of frozen pelleted mitochondria are suspended in 800 μL of 10 mM Tris-acetate pH 7.0 plus 1 mM CaCl₂, 1 mM MgCl₂, 1 mM MnSO₄. Two μL of protease inhibitor cocktail (Sigma) is added to the mitochondria preparation, and Concanavalin A-coupled lanterns, prepared as described in Example 11, are incubated in the preparation for 1 hour. The lanterns are removed and washed with 10 mM Tris-acetate pH 7.0 buffer. The bound glycoproteins are eluted from the lanterns with 8 M urea buffer.

### EXAMPLE 13

### Synthesis of Polystyrene Sulfonic Acid Lanterns 9

Ten polystyrene lanterns were added to a solution of chloroform (20 mL). After 10 minutes chlorosulfonic acid (4 mL) was added, and the mixture shaken at room temperature for 10 minutes. The lanterns were washed five times with dichloromethane, and dried under vacuum to give polystyrene sulfonic acid lanterns.

### EXAMPLE 14

### Synthesis of Bromopolystyrene Lanterns 7

Thallium acetate (400 mg) was dissolved in 100 mg) of dry DCM, yielding a yellow solution with some white precipitate. Fifty lanterns (145 μM×50; 7.25 mmmol) were added into the solution and left shaking for one hour. Bromine (0.5 mL, 9.757 mmol) was added in 0.1 mL portions over a period of 15 minutes. The reaction mixture was left standing at room temperature for one hour, and the solvent decanted off. Lanterns were washed with the following order of solvents: methanol (×3), DCM (×3), warm solution of 20% water in THF (×3), methanol (×2) and DCM (×3). The lanterns were air-dried for one hour under high vacuum (at 25° C.) for 4 hours to yield bromopolystyrene lanterns.

### EXAMPLE 15

### Synthesis of Isocyanate Polystyrene Lanterns 4

Triphosgene (198 mg) was dissolved in dry DCM (10 mL) and gently stirred under nitrogen. Twelve aminomethylated polystyrene lanterns (TFA salt) were added, and left stirring under nitrogen for 10 minutes. The mixture was cooled in an ice bath and after 15 minutes diisopropylethylamine (0.2 mL) was added slowly. A further 0.2 mL of diisopropylethylamine was added, and the mixture stirred under nitrogen for 30 minutes. The mixture was allowed to warm to room temperature, and stirring was continued for 6 hours. The solution was decanted, and the lanterns washed with DCM (×3), THF (×3), DCM (×3) to yield isocyanate polystyrene lanterns.

### EXAMPLE 16

### Synthesis of High-Loading Aminomethylated Polystyrene Lanterns 1

N-Hydroxymethylphthalimide (120 g) was dissolved in 20% trifluoroacetic acid/DCM (2500 mL) and methanesulfonic acid (125 mL) was added 5100 Polystyrene lanterns were added, and the mixture was shaken gently for 24 hours. The lanterns were washed with 20% trifluoroacetic acid/DCM, DCM (×2) and methanol. A solution of 5% hydrazine/methanol (3000 mL) was added, and the mixture refluxed for 18 hours. The lanterns were washed with hot methanol (×4), 1% trifluoroacetic acid/DCM, and DCM. Drying under vacuum yielded high-loading aminomethylated polystyrene lanterns (TFA salt). The loading was 80 micromole/lantern.

### EXAMPLE 17

### Synthesis of Palmitic Acid-Derivatised Lanterns

A 0.1 M solution of palmitic acid/diisopropylcarbidimide/1-hydroxybenzotriazole in DMF is added to 10 high-loading aminomethylated polystyrene lanterns, prepared as described in Example 16, and reacted for 3 hours. The lanterns are washed with DMF (×3) and DCM (×3) before been dried under vacuum for 18 hours.

### EXAMPLE 18

### Use of Palmitic Acid-Derivatised Lanterns for Affinity Purification of Mitochondrial Hydrophobic Proteins

Approximately 100 μL of frozen pelleted mitochondria are suspended in 800 μL of 5 M NH₄OH/10% methanol. Two μL of protease inhibitor cocktail (Sigma) are added to the mitochondria preparation, and palmitic acid-derivatised lanterns, prepared as described in Example 13, are incubated in the preparation for 1 hour. The lanterns are removed and washed with 10% methanol in water. The bound hydrophobic proteins are eluted from the lanterns with 8 M urea buffer.

### EXAMPLE 19

### Synthesis of 2-Chlorotrityl Alcohol Lanterns 14

Aluminium chloride (0.79 g) was mixed with DCM (10 mL), and 20 polystyrene lanterns were added. 2-Chlorobenzoyl chloride (1050 mg) was added, and the mixture stirred for 7 minutes, followed by 3 hours with no stirring. The lanterns were washed with methanol, 20% water in THF (×2), methanol (×2), and DCM (×3). The lanterns were suspended in 0.8 M sodium borohydride in THF, and 2 drops of methanol added. After reacting at room temperature for 18 hours, the lanterns were washed with methanol, 20% water in THF, 20% 2MHCl in THF, 20% water in THF, methanol (×2) and DCM (×3). The lanterns were dried under vacuum for 3 hours to yield 2-chlorotrityl alcohol polystyrene lanterns.

### EXAMPLE 20

### Synthesis of REM Linker Lanterns 15

Ten Hydroxymethyl D-series Lanterns (initial specified loading, 36 μmol) were treated with 5 mL of a solution of DIEA (500 μL, 2870 μmol, 8 mole equivalents) in DCM. To the mixture was added acryloyl chloride (260 μL, 2870 μmol, 8 mole equivalents). The reaction was allowed to stand at art for 4 hours. The reagent solution was decanted and the Lanterns washed with DMF (3×3 min) and DCM (3×3 min).

### EXAMPLE 21

### Synthesis of Morpholino Lanterns 16

A 0.1 M solution of morpholine in dichloromethane (5 mL) was added to 10 SPPSDREM lanterns and reacted overnight at room temperature. The reagent solution was decanted and the Lanterns washed with DMF (3×3 min) and DCM (3×3 min).

### EXAMPLE 22

### Synthesis of Triphenylphosphine Lanterns 11

Under an atmosphere of nitrogen, a 0.1 M solution of chlorodiphenylphosphine in dry THF 5 mL) was added to 10 lithiated polystyrene lanterns 6. After reaction for 3 hrs, the lanterns were washed with dichloromethane (2×10 mins), followed by hot 20% H₂O/THF (1 hour), and soaked in 20% H₂O/THF overnight, then soaked in dichloromethane (30 min), and air dried.

### EXAMPLE 23

### Synthesis of Dimethyl m-isopropenyl Benzyl Isocyanate Lanterns 17

A mixture of Styrene and Dimethyl m-isopropenyl benzyl isocyanate styrene (ratio 3:1) (48 mL) was mixed with methanol (112 mL). The mixture was then poured into a bottle containing 1000 Lanterns. The mixture was purged with nitrogen for 20 minutes. The bottle was capped tightly and then placed in the irradiator machine. The contents were irradiated at a dose rate of 1.6 kGy per hour for 7 hours with occasional shaking of the bottle. The bottle was removed, the styrene monomers drained and the contents washed with dichloromethane (5×10 minutes). The Lanterns were removed and air dried.

### EXAMPLE 24

### Synthesis of 4-methylstyrene/α-methylstyrene Grafted Lanterns

A mixture of Styrene and α-methylstyrene (ratio 1:1) (48 mL) was mixed with methanol (112 mL). The mixture was then poured into a bottle containing 1000 Lanterns. The mixture was purged with nitrogen for 20 minutes. The bottle was capped tightly and then placed in the irradiator machine. The contents were irradiated at a dose rate of 1.6 kGy per hour for 7 hours with occasional shaking of the bottle. The bottle was removed, the styrene monomers drained and the contents washed with dichloromethane (5×10 minutes). The Lanterns were removed and air dried.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

1. An activated modular grafted polymeric surface, in which a polymer is bound, covalently or via an activating agent, to a reagent selected from the group consisting of triphenylphosphine, a reductant or oxidant, a chelating metal, a scavenger, a ligand or a catalyst is bound to the grafted polymeric surface, with the provisos that
(a) when the activating moiety is sulphonate, the polymer is not an α,β,β-trifluorostyrene-grafted polymer or a polyalkadiene;
(b) when the polymer is glycidyl methacrylate-grafted polyethylene, the activating moiety is not a 2-fluorofluoroaniline, a 2,4-fluorofluoroaniline,
(c) when the polymer is polypropylene, the activating moiety is not an aziridine;
(a) when the activating moiety is amino, the polymer is not polyethylene glycol-grafted polystyrene or polystyrene-grafted polyethylene; and
(b) when the activating moiety is aminoalkyl, the polymer is not polystyrene-grafted polytetrafluoroethylene.

2. An activated modular grafted polymeric surface, according to claim 1, in which the activating moiety is aldehyde, carboxylate, amino, hydroxide, biotin, thiol, hydrazino, or isocyanate,
with the provisos that
(c) the activating moiety is not an aniline or an aziridine;
(d) when the activating moiety is amino, the polymer is not polyethylene glycol-grafted polystyrene; and
(e) when the activating moiety is aminoalkyl, the polymer is not polystyrene-grafted polytetrafluoroethylene.

3. An activated modular grafted polymeric surface, in which a polymer is bound, covalently or via an activating agent, to a reagent selected from the group consisting of triphenylphosphine, a reductant or oxidant, a chelating metal, a scavenger, a liqand or a catalyst is bound to the grafted polymeric surface, when used in a method of affinity capture, presentation or preparation of a biomolecule.

4. An activated modular grafted polymeric surface according to any one of claims 1 to 3, in which the polymer is an optionally-substituted polyolefin, silicone polymer, polyurethane, polyamide, polyester, formaldehyde resin, polycarbonate, polyoxymethylene, polyether, or epoxy resin, or a co-polymer comprising any of these.

5. An activated modular grafted polymeric surface according to any one of claims 1 to 3, in which the polymer is a co-polymer of polyethylene and polypropylene (PMA) or a modified branched polyolefin.

6. An activated modular grafted polymeric surface according to claim 5, in which the polymer is a modified branched polyolefin, or a derivative, blend or copolymer thereof, modified by graft polymerization.

7. An activated modular grafted polymeric surface according to claim 5, in which the branched polyolefin is a polyalkylalkene.

8. An activated modular grafted polymeric surface according to claim 6, in which the polyalkylalkene is poly-(4-methylpentene-1).

9. An activated modular grafted polymeric surface according to any one of claims 1 to 8, in which the graft polymerisation is gamma-irradiation graft polymerisation, ozone-induced graft polymerisation, plasma-induced graft polymerisation, UV-initiated graft polymerisation or chemical-initiated graft polymerisation.

10. An activated modular grafted polymeric surface according to claim 6 or claim 9, in which the graft polymer is selected from the group consisting of polyvinyls, polyvinylalcohols, polystyrenes, poly-α-methylstyrenes, polyacrylates, polymethacrylates, polyacrylamides, polyethylkene glycols, polylactic acids, and derivatives, blends and copolymers thereof.

11. An activated modular grafted polymeric surface according to claim 10, in which the graft polymer is polystyrene.

12. An activated modular grafted polymeric surface according to claim 10, in which the graft polymer is a co-polymer of poly-4-methylstyrene and poly-α-methylstyrene.

13. An activated modular grafted polymeric surface according to claim 10, in which the graft polymer is a co-polymer of poly-4-bromostyrene and poly-α-methylstyrene.

14. An activated modular grafted polymeric surface according to claim 10, in which the graft polymer is a co-polymer of polystyrene and poly-dimethyl m-isopropenyl benzyl isocyanate styrene.

15. An activated modular grafted polymeric surface according to claim 10, in which the graft polymer is a co-polymer of polydimethylacrylamide and poly-dimethyl m-isopropenyl benzyl isocyanate styrene.

16. An activated modular grafted polymeric surface according to claim 10, in which the graft polymer is a polyvinylalcohol.

17. An activated modular grafted polymeric surface according to claim 10, in which the graft polymer is polyacrylic acid.

18. An activated modular grafted polymeric surface according to any one of claims 1 to 17, in which the reagent is a chelating metal selected from is nickel and calcium.

19. An activated modular grafted polymeric surface according to any one of claims 1 to 17, in which the reagent is a scavenger which is a nucleophilic group.

20. An activated modular grafted polymeric surface according to claim 19, in which the nucleophilic group is aminomethyl or hydrazino.

21. An activated modular grafted polymeric surface according to any one of claims 1 to 17, in which the reagent is a scavenger which is an electrophilic group.

22. An activated modular grafted polymeric surface according to claim 21, in which the electrophilic group is isocyanate, or benzaldehyde.

23. An activated modular grafted polymeric surface according to any one of claims 1 to 17, in which the reagent is a ligand capable of Schiff base formation.

24. An activated modular grafted polymeric surface according to any one of claims 1 to 17, in which the reagent is a catalyst which is dimethylaminopyridine.

25. An activated modular grafted polymeric surface according to claim 18, in which the graft is polyacrylic acid and the reagent is a chelating metal.

26. An activated modular grafted polymeric surface according to claim 1 or claim 3, in which the activating moiety is aldehyde, carboxylate, amino, hydroxide, biotin, thiol, hydrazino, or isocyanate.

27. An activated modular grafted polymeric surface according to any one of claims 1 to 26, in which the activating moiety is aldehyde.

28. An activated modular grafted polymeric surface according to any one of claims 1 to 27, in which one or more spacer sequences, which may be the same or different, is present between the activating moiety and the grafted polymer.

29. An activated modular grafted polymeric surface according to claim 18, in which the reagent has a structure selected from the group consisting of structures 1 to 8 and 11 to 17 set out in Table 1.

30. An activated modular grafted polymeric surface according to claim 18, selected from the group consisting of
(a) a benzaldehyde polystyrene lantern;
(b) a benzaldehyde polystyrene lantern, coupled to streptavidin or horseradish peroxidase; and
(c) a nickel-chelating polyacrylic acid gear.

31. An activated modular grafted polymeric surface according to any one of claims 17 to 29, in which the reagent is capable of binding an amine compound capable of Schiff base formation.

32. An activated modular grafted polymeric surface according to claim 31, in which the amine compound is a biotinylated molecule, a protein, a peptide, a lectin, an oligonucleotide, a sugar or an enzyme.

33. An activated modular grafted polymeric surface according to claim 32, in which the biotinylated molecule is a peptide, protein, oligonucleotide, lipid or sugar.

34. An activated modular grafted polymeric surface according to claim 32, in which the protein is streptavidin.

35. An activated modular grafted polymeric surface according to claim 32, in which the enzyme is horseradish peroxidase.

36. An activated modular grafted polymeric surface according to any one of claims 1 to 3, in which the polymer is a modified branched polyolefin, or a derivative, blend or co-polymer thereof, modified by graft polymerization.

37. An activated modular grafted polymeric surface according to claim 36, in which the branched polyolefin is a polyalkylalkene.

38. A polymeric surface according to claim 37, in which the polyalkylalkene is poly-(4-methylpentene-1).

39. A grafted polymeric surface according to any one of claims 36 to 38, in which the graft polymerisation is gamma-irradiation graft polymerisation, ozone-induced graft polymerisation, plasma-induced graft polymerisation, UV-initiated graft polymerisation or chemical-initiated graft polymerisation.

40. A grafted polymeric surface according to any one of claims 36 to 39, in which the graft polymer is selected from the group consisting of polyvinyls, polystyrenes, poly-α-methylstyrenes, polyvinylalcohols, polyacrylates, polymethacrylates, polyacrylamides, polyethylkene glycols, polylactic acids, and derivatives, blends and co-polymers thereof.

41. A grafted polymeric surface according to any one of claims 1 to 40, in a modular three-dimensional form.

42. A grafted polymeric surface according to claim 41, in which the modular three-dimensional form is a lantern, crown, gear, pin, puck, disc, bead, microtitre plate or sheet.

43. A method of affinity capture, presentation or preparation of a biomolecule, comprising the step of exposing the biomolecule or a precursor thereof to an activated modular grafted polymeric surface according to any one of claims 1 to 42.

44. A method according to claim 43, in which the biomolecule is selected from the group consisting of proteins, oligonucleotides, nucleic acids, peptides, and lectins.
